(19) United States
(12) Patent Application Publication
(10) Pub. No.: US 2022/0062147 A1
(43) Pub. Date: Mar. 3, 2022

(54) Skin Care Formulation for Treatment of Cellulite

(71) Applicant: BOTTLED SCIENCE LIMITED, London (GB)

(72) Inventor: Piers RAPER, Richmond (GB)

(21) Appl. No.: 17/465,461

(22) Filed: Sep. 2, 2021

Related U.S. Application Data
(60) Provisional application No. 63/074,103, filed on Sep. 3, 2020.

(51) Int. Cl. A61K 8/66; A61K 8/65; A61K 8/67; A61Q 19/06; A61K 8/02

(52) U.S. Cl. CPC A61K 8/66; A61K 8/65; A61K 8/676; C12Y 115/01001; A61K 8/022; A61K 2800/92; A61Q 19/06

(57) ABSTRACT

The present invention relates to a skin care formulation that is suitable for the treatment of cellulite.

### CROSS-REFERENCE TO RELATED APPLICATION

This application claims benefit of U.S. Provisional Application No. 63/074,103 filed Sep. 3, 2020, the complete disclosure of the prior application is hereby incorporated herein by reference in its entirety for all purposes.

### FIELD OF INVENTION

The present invention relates to a skin care formulation that is particularly useful in the treatment of cellulite.

### BACKGROUND

Gynoid lipodystrophy, or cellulite, affects about 85% of the global female adult population. It is one of the most common skin related concerns and can have an impact on an individual's self-image and body confidence. Cellulite causes alternations in the topography of the skin, such as small depressions and dimples. Cellulite predominantly forms on the legs, buttocks and sometimes abdomen, and is widely described as having an “orange peel” like skin appearance.

Human skin is made up of three distinct layers which include the epidermis, dermis, and hypodermis. The hypodermis contains the adipose tissue (fat) which is composed of adipocytes and is anchored by fibrous connective tissue to the dermis above and the anterior fascia of the muscle below. In skin affected by cellulite the adipocytes in the hypodermis containing fat cells herniate and this causes pressure towards the skin surface and substructure. The connective tissue between the adipocytes restrain this pressure and this creates the classic “dimple” effect and skin fibrosis. This also has the effect of restricting the blood flow through the small capillaries in skin which exacerbates the problem.

Cellulite usually starts forming after puberty and is caused and exacerbated by a number of factors such as reduced dermal integrity, fibrosis, fat hypertrophy, fluid retention, toxin build up, metabolic issues, reduction in or loss of skin elasticity, hormonal impacts, poor circulation and weak connection tissue.

Several topical gels and creams are known that allege to treat cellulite. However, their effectiveness is largely unproven. Minimally invasive medical treatments can also be used to treat cellulite, for example, Cryolipolysis (colloquially known as “fat freezing”), which involves freezing fat cells to kill them and excreting the fat released. This procedure is time consuming and requires several treatments, each of which is expensive. Cellulase is another minimally invasive medical treatment for cellulite that involves inserting a laser under the surface of the area to be treated and using the laser to disrupt fat cells. However, this course of treatment lasts 12 months, is painful and expensive. Finally, liposuction can be used to treat cellulite, however, this is a risky medical procedure that is expensive, painful and has a slow recovery.

In all of the above treatments the fat removal is uneven and after healing the untreated areas look worse and “patchy”. Further medical methods for treating cellulite are being developed such as Qwo (collagenase clostridium histolyticum-aaes) injections from Endo International, however, as with the above methods, these are expensive and painful. Thus, there is a need for an inexpensive and non-invasive treatment of cellulite.

Food supplement formulations are known that allege to treat cellulite. However, they all focus on increasing metabolic rate, usually with caffeine and include a diuretic, which does little specific to the skin. In fact, the caffeine boost can make the consumer shaky without affecting metabolism local to the cellulite and the diuretic simply increases urine flow without removing fluid from the areas of cellulite.

All of the known procedures or supplements that allege to treat cellulite focus on one specific cause of cellulite. However, cellulite is known to be a multifactorial condition and therefore requires a treatment that addresses several of these factors. The present invention provides such a treatment. Specifically, the present invention provides a skin care formulation that is administered orally, preferably as a beverage, and thus works from the inside of the body to target the multiple issues associated with cellulite such as oxidative damage, hormonal changes, fibrosis, stress, water retention and poor detoxification as well as improving circulation, metabolism, skin elasticity and the integrity of the dermal matrix and septae to prevent fat hypotrophy. The present invention is advantageous over topical creams and gels as it works from the inside and is therefore able to support skin all over the body. Accordingly, the present invention is effective whether the cellulite is located on the legs buttocks or abdomen.

### SUMMARY OF INVENTION

In a first aspect, the present invention provides a skin care formulation comprising superoxide dismutase, collagen and vitamin C or a derivative thereof.

In a preferred first aspect, the skin care formulation is in the form of a liquid composition and a solid composition. In a further preferred first aspect, the solid composition is a powder.

In a second aspect, the present invention provides a method of treating cellulite comprising administering an effective amount of a skin care formulation according to the first aspect to a subject in need thereof.

The present invention also provides a solid composition for a skin care beverage comprising superoxide dismutase and one or more further anti-inflammatory compound(s). Preferably, the solid composition is a powder.

The present invention also provides a liquid composition for a skin care beverage comprising collagen, vitamin C or a derivative thereof, and L-cysteine or a derivative thereof.

The present invention also provides a skin care beverage comprising the solid composition provided hereinabove (preferably as a powder), the liquid composition provided hereinabove and a liquid vehicle. Preferably, the liquid vehicle is water.

The present invention also provides a method of preparing a skin care beverage wherein the method comprises mixing the solid composition provided hereinabove (preferably as a powder), the liquid composition provided hereinabove and a liquid vehicle, and agitating the resulting mixture. Preferably, the liquid vehicle is water.

The present invention also provides a method of treating cellulite comprising administering an effective amount of the skin care beverage provided hereinabove or prepared as provided hereinabove to a subject in need thereof.

The present invention also provides a skin care product comprising:
(i) a solid composition provided hereinabove; and
(ii) a liquid composition provided hereinabove.

The present invention also provides use of the solid composition provided hereinabove for treating cellulite.

The present invention also provides use of the liquid composition provided hereinabove for treating cellulite.

The present invention also provides use of the skin care beverage provided hereinabove or prepared as provided hereinabove for treating cellulite.

The present invention also provides use of the skin care product provided hereinabove for treating cellulite.

The present invention also provides a method of treating cellulite comprising administering an effective amount of the solid composition provided hereinabove to a subject in need thereof.

The present invention also provides a method of treating cellulite comprising administering the liquid composition provided hereinabove to a subject in need thereof.

The present invention provides a method of treating cellulite comprising administering the skin care product provided hereinabove to a subject in need thereof.

### BRIEF DESCRIPTION OF FIGURES

FIG. 1 shows that when tested in clinical trials the bioactive form of superoxide dismutase used in the skin care formulation according to the present invention significantly reduces subcutaneous fat at the gluteal fold (left-hand chart) and at the femoral fold (right-hand chart).

FIG. 2A and FIG. 2B show an area of an individual's skin affected by cellulite before and after treatment with the skin care formulation according to the present invention.

FIGS. 3A-C show images of an area of an individual's skin affected by cellulite before (left-hand side) and after (right-hand side) treatment with the skin care formulation according to the present invention.

The patent or application file contains at least one drawing executed in color. Copies of this patent or patent application publication with color drawing(s) will be provided by the Office upon request and payment of the necessary fee.

### DETAILED DESCRIPTION

Hereinafter, the present invention will be described in further detail with reference to suitably preferred embodiments. Any combination of the preferred ingredients and amounts herein may be present in the formulation, and the scope of the invention is not limited to the specific combinations and amounts of ingredients disclosed.

Skin Care Formulation

The present invention provides a skin care formulation comprising superoxide dismutase, collagen and vitamin C or a derivative thereof. The skin care formulation according to the present invention may be used in a method of treatment such as a method for treating cellulite.

To be effective in the treatment of cellulite all of the ingredients in the formulation according to the present invention have to be absorbed by the body at the correct speed and concentration. If all of the ingredients were formulated in a solid (e.g. in a tablet or capsule) then a large number of tablets or capsules (e.g. about 30) would be required as it is not possible to formulate all of the ingredients in a single tablet or capsule due to ingredient stability and/or reactivity between ingredients. Taking a large number of tablets regularly (e.g. each day) is not desirable for a subject. In addition, if all of the ingredients were formulated in a solid then the amount of each ingredient in the solid would have to be at least about 3 times greater than the required dose of the ingredient because typically, when the ingredients are administered in solid form only about 35% of the active material would be absorbed by the body.

It is desirable for the ingredients to be formulated as a liquid that can be consumed orally, e.g. as a beverage, in order to enable the body to absorb all of the ingredients at the rate and concentration that provides efficacy. However, formulating all of the ingredients in a liquid has various drawbacks. For example, some ingredients, such as superoxide dismutase, are unstable in solution. Therefore, formulating these ingredients in a liquid would not provide a formulation that is effective in the treatment of cellulite as the unstable ingredients would decompose, thereby losing effectiveness. Similarly, some ingredients may react with each other if they are in a liquid composition. This would result in the loss of the active ingredients from the formulation and the formation of reaction products that may have no effect in the treatment of cellulite, or that have a detrimental effect. Furthermore, some ingredients have a bitter taste and therefore including them in a liquid that is intended for oral administration (preferably as a beverage) results in a treatment that is not palatable to the subject.

The present inventors have solved the above mentioned problems by formulating the formulation according to the present invention as a solid composition and a liquid composition. Accordingly, in a preferred embodiment, the skin care formulation according to the present invention is in the form of a solid composition and a liquid composition, and optionally also a capsule. Preferably, the solid composition is a powder. Preferably, the powder and liquid composition are administered concurrently as a beverage.

As used herein the term “powder” refers to a dry solid comprising fine particles typically prepared by grinding, crushing or thermal decomposition of a solid substance, or by the deposition of solids from the liquid or vapour phase. As used herein, the term “capsule” refers to a solid wherein the active ingredients are enclosed in a hard or soft shell. Preferably, the shell comprises gelatin or cellulose, preferably hydroxypropyl methyl cellulose (HMPC). Preferably, the hydroxypropyl methyl cellulose is combined with titanium dioxide.

Superoxide dismutase (SOD) is an enzyme that catalyses the dismutation (also referred to as disproportionation) of superoxide radicals (i.e. O₂⁻) into molecular oxygen (i.e. O₂) and hydrogen peroxide (H₂O₂). It therefore plays a key role in the cellular antioxidant defence system. In addition to being an antioxidant, superoxide dismutase may also function as an anti-inflammatory agent. Without being bound in this regard, the inventors believe that in the present invention, the superoxide dismutase (SOD) advantageously boosts the endogenous antioxidant defence mechanisms, which reduces hypertrophy of fat cells and corruption of connective tissues associated with oxidative stress.

Superoxide dismutase is naturally sourced from melons. The superoxide dismutase used in the present invention is preferably sourced from a cantaloupe melon grown in the south of France. Preferably, the superoxide dismutase used in the present invention is commercially available under the trade name “Dimpless®” from Robertet, France.

The present inventors have advantageously found that combining superoxide dismutase with collagen and vitamin C (or a derivative thereof) in the skin care formulation according to the present invention provides a formulation that is more effective in the treatment of cellulite than if each ingredient was used alone. Specifically, the combination of superoxide dismutase with collagen and vitamin C (or a derivative thereof) provides a powerful formulation that can rejuvenate and improve the quality of the hypodermal tissue, and reduce the fibrosis affecting the connective tissues to a greater extent than when superoxide dismutase, collagen or vitamin C are administered individually.

The superoxide dismutase used in the present invention is unstable in solution, for example in water. Accordingly, the superoxide dismutase is present in the solid composition (preferably a powder composition) of the skin care formulation and when said composition is mixed with a liquid vehicle, for example water, to provide a beverage as described hereinbelow the beverage is preferably administered within about 5 minutes of preparation, preferably within about 3 minutes of preparation, more preferably within about 1 minute of preparation in order to ensure the superoxide dismutase maintains efficacy and is absorbed.

Preferably, the superoxide dismutase is present in the solid composition in an amount of from about 1 to about 15 wt %, preferably from about 2 to about 10 wt %, more preferably from about 3 to about 8 wt % of the solid composition. Without being bound in this regard, the inventors believe that in this amount the superoxide dismutase advantageously visibly lowers fat nodes.

Collagen is the main structural protein in various connective tissues in the human body and provides tensile strength and elasticity to the dermal matrix. The most common type of collagen in skin is type I (or type “i”) collagen. One of the major causes of cellulite is the breakdown and interruption of the dermal matrix leading to uneven and weakened dermal tissue, which results in bulging of subcutaneous fat. Accordingly, preventing or repairing such a breakdown of the dermal matrix may be beneficial in the treatment of cellulite.

Preferably, the collagen is a type “i” collagen peptide. Without being bound in this regard, the inventors believe that this type “i” collagen peptide specifically triggers correct connective collagen formation. This is advantageous in the treatment of cellulite as this mechanism (i.e. correct connective collagen formation) replaces lost collagen and rebuilds the existing dermal matrix. In addition, the collagen also prevents collagen fibrosis.

The collagen used in the present invention is suitably derived from animal or marine sources. In particular, the collagen is preferably hydrolysed collagen, preferably with a molecular weight of about 500 to 20000 Da. A suitable collagen is available under the trade name Verisol® B and is available from Gelita AG, Germany.

Preferably, the collagen is present in the liquid composition, preferably in an amount of from about 1000 to about 6000 mg/15 mL, preferably from about 1500 to about 5000 mg/15 mL, more preferably from about 2000 to about 4000 mg/15 mL of the liquid composition.

Vitamin C plays a role in the formation of new collagen and the fibroblast response to peptide feedback mechanism. The present inventors have advantageously found that combining vitamin C or a derivative thereof with collagen in the liquid composition according to the present invention enhances collagen synthesis in the dermal matrix and reduces fibrosis further than collagen alone. Combining collagen and vitamin C also leads to an improvement of skin hydration, elasticity and firmness. Accordingly, this combination is useful in treating cellulite.

The term “vitamin C” herein refers to ascorbic acid. The vitamin C derivative may be an ascorbate salt, such as a salt with one or more cations from Group IA elements, Group IIA elements, or quaternary ammonium salts. A suitable ascorbate salt is calcium ascorbate, which is available under the name Ester-C® from The Ester C Company, Bohemia, N.Y., USA. This salt is kinder to the stomach than ascorbic acid. In other embodiments the vitamin C derivative may be an ester of ascorbic acid such as an alkyl, alkoxy, or phosphate ester or polyphosphate ester. A suitable ester is ascorbate-2-diphosphate or ascorbate-2-triphosphate or mixtures thereof, for example as available under the Registered Trade Mark STAY-C. Preferably, the vitamin C derivative comprises, consists of, or consists essentially of calcium ascorbate.

Preferably, the vitamin C or derivative thereof is present in the liquid composition in an amount of from about 200 to about 800 mg/15 mL, preferably from about 300 to about 700 mg/15 mL, more preferably from about 400 to about 600 mg/15 mL of the liquid composition.

Preferably, the weight ratio of collagen to vitamin C (preferably as calcium ascorbate) is from about 8:1 to about 3:1, preferably about 5:1.

Preferably, the skin care formulation further comprises a dandelion root extract, bromelain, a milk thistle extract, a horse chestnut extract, one or more vitamin B complex(es) or derivative(s) thereof, or combinations thereof.

Unless stated otherwise, all extracts used herein are solvent extracts prepared by solvent extraction using a mixture of water and ethanol. Preferably, the solvent mixture used is a 50:50 mix, preferably a 70:30 mix, more preferably an 80:20 mix of water and ethanol respectively.

Without being bound in this regard, it is believed that dandelion root extract advantageously helps reduce water retention and detoxifies the liver from excess toxins and hormones, and that the horse chestnut extract reactivates circulation, reduces swelling and minimises water retention. Swollen and heavy legs and capillary fragility are widespread symptoms of cellulite, therefore, dandelion extract and horse chestnut extract may have a beneficial effect in the treatment of cellulite. Horse chestnut extract can also function as an anti-inflammatory agent.

Preferably, the dandelion root extract is present in the liquid composition, preferably in an amount of from about 100 to about 700 mg/15 mL, preferably from about 200 to about 600 mg/15 mL, more preferably from about 300 to about 500 mg/15 mL of the liquid composition. Preferably, the horse chestnut extract is present in the liquid composition, preferably in an amount of from about 50 to about 600 mg/15 mL, preferably from about 100 to about 500 mg/15 mL, more preferably from about 150 to about 400 mg/15 mL of the liquid composition. Preferably, the horse chestnut extract comprises 10-50% aescin, more preferably 10-40%, more preferably 20% aescin. Aescin is a mixture of saponins that may have anti-inflammatory, vasoconstrictor and vasoprotective effects. In an alternative preferred embodiment, the skin care formulation further comprises aescin, preferably in the form of a horse chestnut extract.

In an alternative preferred embodiment, the dandelion root extract and/or the horse chestnut extract are present in a capsule that is administered concurrently or sequentially with the skin care formulation. Advantageously, formulating the dandelion root extract and horse chestnut extract in a capsule improves the palatability of the skin care formulation, especially when the powder and liquid composition are administered as a beverage.

Bromelain is an enzyme extract that may be derived from the fruit or stem of pineapple. Preferably, the bromelain used in the present invention is stem bromelain, i.e. derived from the stem of pineapple. As well as being a powerful anti-inflammatory agent, bromelain prevents and actively lowers fluid retention and improves digestive function. Preferably, the bromelain used in the present invention has a strength (i.e. enzyme potency) of 100 gdu/g. Preferably, the bromelain is present in the solid composition, preferably in an amount of from about 2 to about 30 wt %, preferably from about 5 to about 25 wt %, more preferably from about 8 to about 20 wt % of the solid composition. Preferably, when the bromelain is present in the solid composition, the solid composition is a powder.

Milk thistle (silybum marianum) is a herb native to southern Europe. Traditionally, milk thistle extract is made from the milk thistle seeds, which contain silymarin. Accordingly, the predominant compound in milk thistle extract is typically silymarin. Silymarin is a complex mixture of polyphenolic molecules, including seven closely related flavonolignans (silybin A, silybin B, isosilybin A, isosilybin B, silychristin, isosilychristin, silydianin) and one flavonoid (taxifolin). As well as being an anti-inflammatory agent, milk thistle extract is capable of removing toxins from the body and supporting liver function. Preferably, the milk thistle extract is present in the solid composition, and preferably in an amount of from about 30 to about 90 wt %, preferably from about 40 to about 80 wt %, more preferably from about 50 to about 60 wt % of the solid composition. Preferably, when the milk thistle extract is present in the solid composition, the solid composition is a powder.

Preferably, the milk thistle extract used herein is prepared by solvent extraction using a mixture of water and ethanol as the solvent to provide an extract that is 80% silymarin. Preferably, the solvent mixture used to provide the milk thistle extract is a 50:50 mix, preferably a 70:30 mix, more preferably an 80:20 mix of water and ethanol respectively. Typically, the solvent extract is dried by suitable means (e.g. rotary evaporation) to provide a solid, preferably a powder. Preferably, the milk thistle extract used herein is a solid, preferably a powder.

Preferably, the skin care formulation further comprises silybin A, silybin B, isosilybin A, isosilybin B, silychristin, isosilychristin, silydianin or taxifolin, preferably from a milk thistle extract. In an alternative preferred embodiment, the skin care formulation further comprises silybin A, silybin B, isosilybin A, isosilybin B, silychristin, isosilychristin, silydianin or taxifolin, wherein the flavonolignans or flavonoid are not from a milk thistle extract.

Preferably, the skin care formulation comprises bromelain and milk thistle extract. Preferably, the bromelain and milk thistle extract are both present in the solid composition (preferably as a powder) of the formulation. Preferably, the bromelain and milk thistle extract are present in a weight ratio of from about 1:6 to about 1:4.

Vitamin B complexes are suitable for improving blood flow. Poor blood flow is reported to be a major cause of cellulite formation due to associated stagnation, fluid retention and poor detoxification. This results in low nutrient delivery and a negative cycle that through continuous increase of adipocytes lowers blood flow even further. Thus, improving blood flow may be beneficial in the treatment of cellulite. Accordingly, the skin care formulation preferably comprises one or more vitamin B complex(es) or derivative(s) thereof. The vitamin B complex or derivative thereof can be any vitamin B complex or derivative thereof that preferably has a beneficial effect in treating cellulite.

Preferably, the vitamin B complex comprises vitamin B3, more preferably niacinamide (also known as nicotinamide). Without being bound in this regard, the inventors believe that in the present invention the vitamin B3 (preferably niacinamide) advantageously improves microcirculation of the skin. More specifically, without being bound in this regard, it is believed that the vitamin B3 (preferably niacinamide) advantageously dilutes the small capillaries and therefore enables increased blood flow to the area of skin affected by cellulite.

Preferably, the vitamin B3 is present in the liquid composition, preferably in an amount of from about 1 to about 200 mg/15 mL, preferably from about 5 to about 100 mg/15 mL, preferably from about 10 to about 50 mg/15 mL of the liquid composition.

Preferably, the vitamin B complex comprises vitamin B1 (also known as thiamine). When present, thiamine is preferably present in the liquid composition, preferably in an amount of from about 1 to about 200 mg/15 mL, preferably from about 5 to about 100 mg/15 mL, preferably from about 10 to about 50 mg/15 mL of the liquid composition. Without being bound in this regard, the inventors believe that in the present invention thiamine is capable of reducing the size of fat cells and plays an important role in liver detoxification.

Preferably, the vitamin B complex comprises a combination of vitamin B3 and vitamin B1, more preferably the vitamin B complex comprises a combination of niacinamide and thiamine. Additional vitamin B complexes, for example vitamin B2, B5, B7, B9 and B12, may also present in the formulation described herein.

Preferably, the skin care formulation further comprises vitamin E or a derivative thereof. Typically, the vitamin E or derivative thereof is selected from alpha- (α-), beta- (β-), gamma- (γ-) or delta (δ-) tocopherol or tocotrienol, acetate or succinate salts thereof. In addition to being a powerful antioxidant, vitamin E and derivatives thereof are also suitable free radical scavengers.

Preferably, the vitamin E or derivative used herein is a solid, preferably a powder. Preferably, the vitamin E or derivative thereof is present in the solid composition, preferably in an amount of from about 5 to about 50 wt %, preferably from about 10 to about 40 wt %, more preferably from about 15 to about 30 wt % of the solid composition. Preferably, when the vitamin E or derivative thereof is present in the solid composition, the solid composition is a powder.

Preferably, the vitamin E derivative is alpha-tocopheryl succinate, more preferably D-alpha tocopheryl succinate (also referred to as RRR alpha tocopheryl succinate). Preferably, the D-alpha-tocopheryl succinate is present in the solid composition, preferably an amount of from about 5 to about 50 wt %, preferably from about 10 to about 40 wt %, more preferably from about 15 to about 30 wt % of the solid composition. Preferably, when the D-alpha-tocopheryl succinate is present in the solid composition, the solid composition is a powder.

The skin care formulation according to the present invention preferably further comprises L-cysteine or a derivative thereof, L-carnitine or a derivative thereof, ascophyllum kelp or combinations thereof.

L-cysteine is the naturally occurring form of the semi-essential amino acid cysteine. Cysteine is a component of the antioxidant glutathione, which is a vital cellular antioxidant that minimizes lipid peroxidation of cell membranes. Preferably, the L-cysteine used in the skin care formulation according to the present invention is N-acetyl-L-cysteine, which is also known simply as acetylcysteine. N-acetyl-L-cysteine (NAC) is a precursor in the formation of glutathione in the body. Without being bound in this regard, the inventors believe that in the present invention N-acetyl-L-cysteine helps replenish intracellular glutathione in the body and increases the activity of the nitric oxide synthase (NOS) enzyme to boost nitric oxide (NO) levels in the body thereby increasing the blood flow to muscles and increasing metabolic rate.

Preferably, the L-cysteine or derivative thereof is present in the liquid composition, preferably in an amount of from about 200 to about 800 mg/15 mL, preferably from about 300 to about 700 mg/15 mL, more preferably from about 400 to about 600 mg/15 mL of the liquid composition. In an alternative preferred embodiment the L-cysteine or derivative thereof is present in a capsule that is administered concurrently or sequentially with the skin care formulation.

Preferably, the skin care formulation further comprises L-carnitine or a derivative thereof. Preferably, the skin care formulation further comprises N-acetyl-L-carnitine. Preferably, the N-acetyl-L-carnitine is present in the liquid composition, preferably in an amount of from about 50 to about 600 mg/15 mL, preferably from about 100 to about 500 mg/15 mL, more preferably from about 150 to about 400 mg/15 mL of the liquid composition. Without being bound in this regard, N-acetyl-L-carnitine is believed to advantageously play a role in metabolism and energy production. More specifically, N-acetyl-L-carnitine is believed to transport long-chain fatty acids into the cells mitochondria to produce energy. Additionally, it is believed that N-acetyl-L-carnitine transports toxins and prevents their accumulation. Toxin build up is a major contributor to cellulite. Accordingly, N-acetyl-L-carnitine is suitable for use in treating cellulite.

Preferably, skin care formulation of the present invention further comprises one or more organic seagreen(s) such as ascophyllum kelp. Preferably, the ascophyllum kelp is present in the liquid composition, preferably in an amount of from about 50 to about 600 mg/15 mL, preferably from about 100 to about 500 mg/15 mL, more preferably from about 150 to about 400 mg/15 mL of the liquid composition. Without being bound in this regard, it is believed that the organic seagreens (preferably ascophyllum kelp) advantageously reduces water retention, counteracts stagnation, stimulates the metabolism and promotes weight loss via lipolysis (i.e. the process of fat break-down). Furthermore, organic seagreens (preferably ascophyllum kelp) are rich in unsaturated fats and therefore have anti-inflammatory and firming properties. In an alternative preferred embodiment, the organic seagreens (preferably ascophyllum kelp) are present in the solid composition of the skin care formulation. Preferably, when the organic seagreens (preferably ascophyllum kelp) are in a capsule that is administered concurrently or sequentially with the skin care formulation.

Preferably, the skin care formulation of the present invention further comprises a natural source of organic sulfur such as methyl sulphonyl methane (MSM). MSM may be used in elastin formation and is believed to prevent collagen fibre cross linking. MSM also functions as an anti-inflammatory agent and regulates osmotic flow making it a powerful detox agent. Preferably, the MSM is present in the liquid composition, preferably in an amount of from about 100 to about 4000 mg/15 mL, preferably from about 250 to about 3000 mg/15 mL, more preferably from about 500 to about 2000 mg/15 mL of the liquid composition.

Preferably, the skin care formulation of the present invention further comprises one or more adaptogen(s) such as holy basil extract. Without being bound in this regard, it is believed that holy basil extract maintains blood glucose levels, supports adrenal function, reduces appetite, sugar cravings and stress (i.e. the negative impact from the hormone cortisol), which are all key issues that lead to and exacerbate cellulite. In a preferred embodiment, the holy basil extract is present in the liquid composition, preferably in an amount of from about 100 to about 1500 mg/15 mL, preferably from about 200 to about 1000 mg/15 mL, more preferably from about 300 to about 800 mg/15 mL of the liquid composition. In an alternative preferred embodiment the holy basil extract is present in a capsule that is administered concurrently or sequentially with the skin care formulation.

Imbalances in estrogen levels lead to increased water retention and reduced collagen and elastin. Therefore, correcting this imbalance may be beneficial in the treatment of cellulite. Preferably, the skin care formulation further comprises one or more compounds(s) that are capable of balancing hormone levels such as red clover extract. Without being bound in this regard, it is believed that red clover extract advantageously rebalances estrogen levels. When present, the red clover extract is present in the liquid composition, preferably in an amount of from about 50 to about 600 mg/15 mL, preferably from about 100 to about 500 mg/15 mL, more preferably from about 150 to about 400 mg/15 mL of the liquid composition. In an alternative preferred embodiment the red clover extract is present in a capsule that is administered concurrently or sequentially with the skin care formulation.

Preferably, the skin care formulation according to the present invention further comprises vitamin A or a derivative thereof. Preferably, the vitamin A derivative is retinol acetate. Without being bound in this regard, it is believed that the retinol acetate advantageously encourages normal skin cell renewal, stimulates fibroblasts for increased collagen production and increased elasticity. Thus, retinol acetate may be beneficial in treating cellulite. When present, the retinol acetate is present in the liquid composition, preferably in an amount of from about 3000 to about 10000 μg/15 mL, preferably from about 3500 to about 8000 μg/15 mL, more preferably from about 4000 to about 6500 μg/15 mL of the liquid composition.

Preferably, the skin care formulation further comprises zinc or a salt thereof, more preferably zinc gluconate. Without being bound in this regard, it is believed that in the present invention zinc gluconate works with vitamin C in the production of collagen. When present, the zinc gluconate is present in the liquid composition, preferably in an amount of from about 1 to about 200 mg/15 mL, preferably from about 5 to about 100 mg/15 mL, preferably from about 10 to about 50 mg/15 mL of the liquid composition.

Preferably, the skin care formulation according to the present invention further comprises copper or a salt thereof, more preferably copper bisglycinate. Without being bound in this regard, it is believed that the copper bisglycinate is advantageously involved in collagen synthesis along with zinc and vitamin C. Copper bisglycinate is also advantageously believed to strengthen dermal integrity and increase skin elasticity. When present, the copper bisglycinate is present in the liquid composition, preferably in an amount of from about 500 to about 8000 μg/15 mL, preferably from about 1000 to about 7000 μg/15 mL, more preferably from about 1000 to about 5000 μg/15 mL of the liquid composition. In an alternative preferred embodiment the copper or salt thereof is present in a capsule that is administered concurrently or sequentially with the skin care formulation.

The skin care formulation according to the present invention further comprises one or more additional agent(s) that may have a beneficial effect in treating cellulite. For example, the skin care formulation can further comprise one or more additional ingredient(s) that are capable of increasing blood flow; one or more additional ingredient(s) that improve osmotic flow; one or more additional ingredient(s) that are capable of improving water retention and/or removing toxins from the body; one or more additional ingredient(s) that are capable of boosting fat metabolism; one or more additional anti-inflammatory ingredient(s); one or more additional ingredient(s) that are capable of re-balancing hormone levels; or one or more additional ingredient(s) that are capable of stimulating collagen production and/or improving skin elasticity.

Preferably, the skin care formulation of the present invention further comprises a liquid vehicle. Suitable liquid vehicles are water, fruit juice and water-based flavoured beverages. Preferably, the liquid vehicle is water. Preferably, the liquid vehicle content is at least about 80%, preferably at least about 85%, more preferably at least 90% of the total weight of the formulation. When the skin care formulation comprises a liquid vehicle (e.g. water) it may be described as a beverage.

As used herein, there term “liquid composition” includes solutions and suspensions. As used herein, the term beverage also includes solutions and suspensions (i.e. microsolids suspended in solution).

As described hereinabove, the skin care formulation according to the present invention may be in the form of a liquid composition and a solid composition. Preferably, the solid composition is a powder. Preferably, the skin care formulation may further comprise a liquid vehicle. Accordingly, in a preferred embodiment, the skin care formulation can be considered as a skin care beverage comprising a powder, a liquid composition and a liquid vehicle.

Preferably, the amount of powder composition present in the skin care beverage is from about 250 mg to about 2500 mg, preferably from about 500 to about 2000 mg, more preferably from about 750 mg to about 1500 mg. Preferably, the amount of liquid composition present in the skin care beverage is from about 5 to about 25 mL, preferably from about 8 to about 20 mL, more preferably from about 12 to about 18 mL. Preferably, the amount of liquid vehicle present in the skin care beverage is from about 50 mL to about 300 mL, preferably from about 80 mL to about 250 mL, preferably from about 100 mL to about 200 mL. Preferably, the volume ratio of the liquid composition to the liquid vehicle is from about 1:2 to about 1:20, preferably from about 1:5 to about 1:15, more preferably about 1:10.

The present invention also provides a method for preparing a skin care beverage as described hereinabove wherein the method comprises mixing the powder composition described hereinabove, the liquid composition described herein above and a suitable liquid vehicle, and agitating the resulting mixture. Preferably, the amount of powder composition used to prepare the skin care beverage is from about 250 mg to about 2500 mg, preferably from about 500 to about 2000 mg, more preferably from about 750 mg to about 1500 mg. Preferably, the amount of liquid composition used to prepare the skin care beverage is from about 5 to about 25 mL, preferably from about 8 to about 20 mL, more preferably from about 12 to about 18 mL. Preferably, the amount of liquid vehicle used to prepare the skin care beverage is from about 50 mL to about 300 mL, preferably from about 80 mL to about 250 mL, preferably from about 100 mL to about 200 mL. Preferably, the volume ratio of the liquid composition to the liquid vehicle is from about 1:2 to about 1:20, preferably from about 1:5 to about 1:15, more preferably about 1:10. Preferably, the liquid vehicle is water, more preferably the water is below room temperature, i.e. at a temperature below about 25° C., preferably below about 20° C., more preferably below about 15° C.

### Method of Treatment

The skin care formulation according to the present invention may be used in a method for treating cellulite. Accordingly, the present invention provides a method of treating cellulite comprising administering an effective amount of the skin care formulation according to the present invention to a subject in need thereof. Preferably, the subject is a human. Preferably, the effective amount of the skin care formulation is from about 50 to about 300 mL/day, preferably from about 70 to about 250 mL/day, more preferably from about 100 to about 200 mL/day.

It will be appreciated that the skin care formulation disclosed herein is administered by oral administration.

Preferably, the treatment is administered daily, preferably for at least about 30 days, preferably for at least about 60 days, more preferably for at least about 90 days. Advantageously, after completing treatment with the skin care formulation according to the present invention, users exhibit firmer skin, smoother skin, more even skin, reduced lumpiness and dimpling, increased skin elasticity and reduction in the appearance of “orange peel”. Accordingly, the skin care formulation according to the present invention is effective in the treatment of cellulite.

The present invention also provides a skin care product comprising the solid composition described hereinabove and the liquid composition described hereinabove. The skin care product according to the present invention may be used in a method for treating cellulite.

As described hereinabove, in a preferred embodiment, the skin care formulation can be considered as a skin care beverage comprising a powder, a liquid composition and a liquid vehicle. Advantageously, formulating the skin care formulation according to the present invention as a beverage allows all of the ingredients therein to be absorbed by the body at the rate and concentration required for efficacy. Thus, the formulation according to the present invention is able to target the multiple specific issues associated with cellulite described hereinabove rather than only one. Conversely, if the formulation according to the present invention was a solid, e.g. a tablet, multiple tablets would be required. Thus, the user would have to consume multiple tablets daily, which is undesirable for the consumer. Furthermore, in tablet form the ingredients would not be absorbed at the correct rate and concentration, which are both essential for optimum efficacy of the skin care product.

Advantageously, when the solid composition is a powder it may be considered as an activator or activating composition. In particular, the powder composition is capable of activating the liquid composition to provide a unique formulation or beverage that is more powerful than the sum of its parts in the treatment of cellulite. Specifically, the combination of superoxide dismutase from the powder composition with collagen and vitamin C (or a derivative thereof) from the liquid composition provides a powerful formulation that can rejuvenate and improve the quality of the hypodermal tissue, and reduce the fibrosis affecting the connective tissues to a greater extent than when superoxide dismutase or collagen and vitamin C are administered individually.

Preferably, the method of treating cellulite provided herein further comprises administering a capsule concurrently or sequentially with the formulation, wherein the capsule comprises dandelion root extract, horse chestnut extract, L-cysteine or a derivative thereof, copper or a salt thereof, red clover extract, ascophyllum kelp, or combinations thereof. Preferably, the capsule is administered less than about 5 minutes, preferably less than about 2 minutes, more preferably less than about 1 minute before or after the formulation.

The skin care formulation according to the present invention further comprises one or more additional components to enhance the formulation, for example one or more flavouring agent(s), colourant(s), sweetener(s), fruit juice concentrate and acidulant(s) may be include in the beverage. Preferably, a flavouring agent is included to enhance the flavour of the formulation. Preferably, natural red cherry and apple juice are used to flavour the formulation.

The skin care formulation according to the present invention may further comprise additional optional components. For example, the formulation may comprise one or more vitamins, minerals, additives, antioxidants, carbohydrate sources, amino acids, trace elements, hyaluronic acid, glucosamine, fruit/vegetable juices, preservatives (such as potassium sorbate), emulsifiers/hydrocolloids, oils, carbonation components and the like.

The present invention will now be described further with reference to the following examples.

### EXAMPLES

### Example 1

In order to demonstrate the efficacy of a formulation according to the present invention in the treatment of cellulite, one of the active ingredients (superoxide dismutase) was administered to subjects with cellulite and the subcutaneous fat was measured after 90 days.

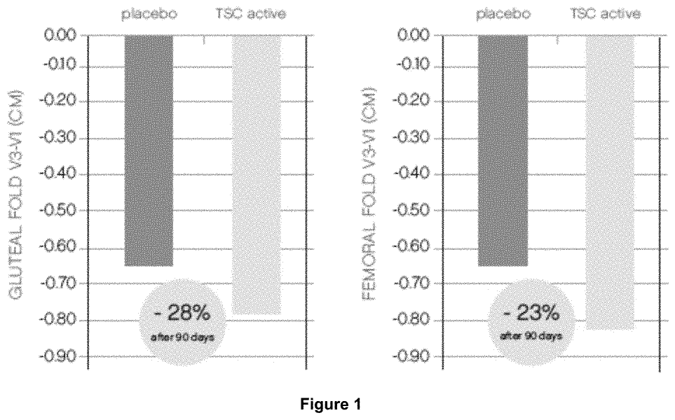

As shown in FIG. 1, there was a 28% reduction in subcutaneous fat compared with the placebo after 90 days at the gluteal fold. Similarly, there was a 23% reduction in subcutaneous fat compared with the placebo after 90 days at the femoral fold.

### Example 2

A powder composition is prepared by combining the following ingredients in a conventional manner.

| Ingredient | Amount (mg) |
|---|---|
| Milk thistle | 650 |
| extract (80%) | |
| Vitamin E | 231 |
| Bromelain | 125 |
| Superoxide Dismutase | 75 |

A liquid composition is prepared by combining the following ingredients in a conventional manner. The ingredients are used either as an aqueous solution or as microsolids suspended in aqueous solution.

| | Amount |
|---|---|
| Ingredient | (mg/15 mL) |
| Collagen | 2500 |
| Methyl sulfonyl methane | 1000 |
| Holy basil extract | 575 |
| N-acetyl-L-cysteine | 500 |
| Vitamin C calcium ascorbate | 500 |
| Dandelion Root Extract | 400 |
| Horse Chestnut extract | 300 |
| (20% aescin) | |
| Organic seagreens | 286 |
| (Ascophyllum kelp) | |
| N-acetyl-L-carnitine | 250 |
| Red clover extract | 250 |
| Thiamine (vitamin B1) | 25 |
| Zinc gluconate | 20 |
| Niacinamide (vitamin B3) | 20 |
| Retinol acetate (vitamin A) | 4.61 |
| Copper bisglycinate | 2 |

The powder composition and the liquid composition prepared above were then combined with 150 mL of water and shaken to prepare a skin care beverage.

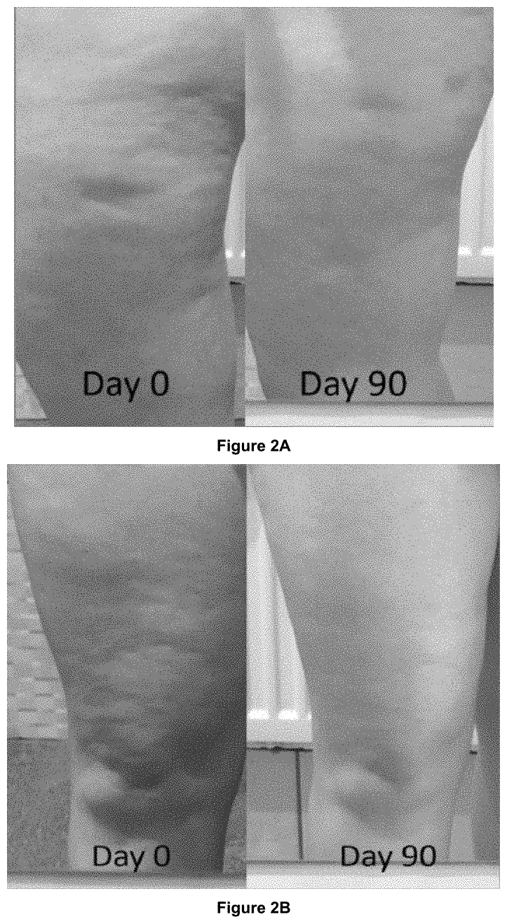

The skin care formulation prepared above was then administered daily to individuals affected by cellulite over a period of 90 days and the results from one individual are shown in FIG. 2. Specifically, after 90 days of treatment there was a significant reduction in the appearance of “orange peel” skin and users reported firmer skin, smoother skin, reduced lumpiness and dimpling, more even skin and increased skin elasticity.

### Example 3

Photographs of individuals affected by cellulite were taken before and after daily administration of the formulation described in Example 2.

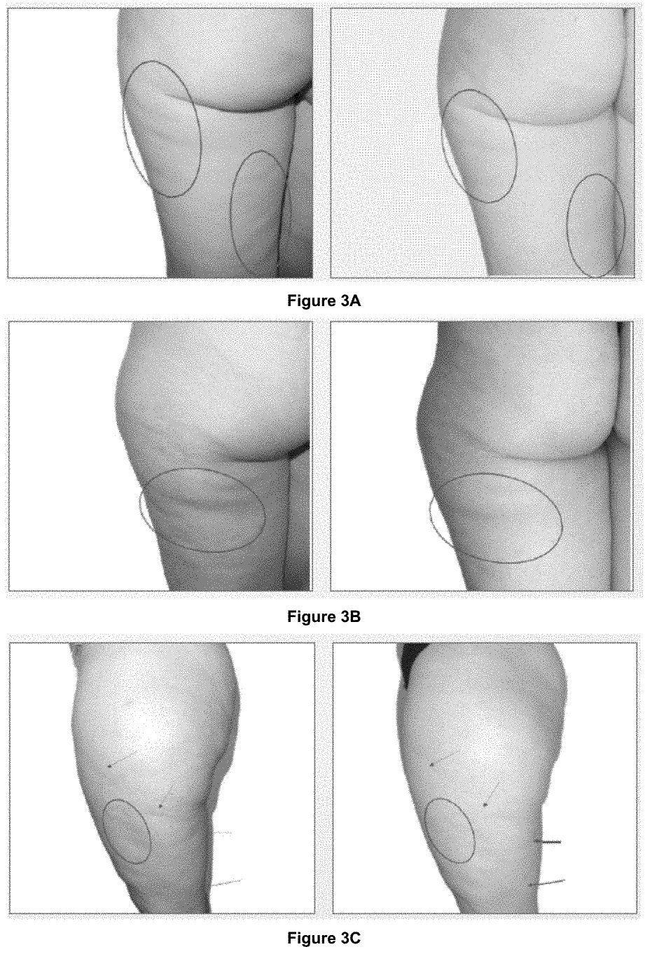

FIGS. 3A-C show areas around the buttocks and thigh of an individual affected by cellulite before and after treatment. After 90 days of treatment there was a significant reduction in the appearance of “orange peel” skin and users reported firmer skin, smoother skin, reduced lumpiness and dimpling, more even skin and increased skin elasticity.

It should be understood that the present invention has been described only by way of example and that modifications of the formulation may be made within the scope of the accompanying claims.

1. A skin care formulation comprising superoxide dismutase, collagen and vitamin C or a derivative thereof.

2. The skin care formulation according to claim 1 further comprising a dandelion root extract, bromelain, a milk thistle extract, a horse chestnut extract, one or more vitamin B complex(es) or derivative(s) thereof, or combinations thereof.

3. The skin care formulation according to claim 1 further comprising L-cysteine or a derivative thereof, L-carnitine or a derivative thereof, ascophyllum kelp, or combinations thereof.

4. The skin care formulation according to claim 1 further comprising a vitamin E complex or derivative thereof, methyl sulfonyl methane, a holy basil extract, a red clover extract, zinc or a salt thereof, a vitamin A complex or a derivative thereof, copper or a salt thereof, or combinations thereof.

5. The skin care formulation according to claim 1 wherein the collagen is hydrolysed collagen.

6. The skin care formulation according to claim 1 wherein the vitamin C or derivative thereof comprises calcium ascorbate.

7. The skin care formulation according to claim 2 wherein the vitamin B complex or derivative thereof is vitamin B3 niacinamide, vitamin B1 or combinations thereof.

8. The skin care formulation according to claim 3 wherein the L-cysteine or derivative thereof is N-acetyl-L-cysteine.

9. The skin care formulation according to claim 3 wherein the L-carnitine or derivative thereof is N-acetyl-L-carnitine.

10. The skin care formulation according to claim 4 wherein the vitamin E complex or derivative thereof is D-alpha tocopheryl succinate.

11. The skin care formulation according to claim 4 wherein the vitamin A complex or derivative thereof is retinal acetate.

12. The skin care formulation according to claim 1 wherein the formulation is in the form of a solid composition and a liquid composition.

13. The skin care formulation according to claim 12 wherein the solid composition is a powder.

14. The skin care formulation according to claim 12 wherein the collagen and vitamin C or derivative thereof are present in the liquid composition.

15. The skin care formulation according to claim 14 wherein the collagen is present in an amount of from about 1000 to about 6000 mg/15 mL of the liquid composition.

16. The skin care formulation according to claim 14 wherein the vitamin C or derivative thereof is present in an amount of from about 200 to about 800 mg/15 mL of the liquid composition.

17. The skin care formulation according to claim 12 wherein the superoxide dismutase is present in the solid composition.

18. The skin care formulation according to claim 17 wherein the superoxide dismutase is present in an amount of from about 1 to about 15 wt % of the solid composition.

19. The skin care formulation according to claim 12 wherein the bromelain is present in the solid composition.

20. The skin care formulation according to claim 19 wherein the bromelain is present in an amount of from about 2 to about 30 wt % of the solid composition.

21. The skin care formulation according to claim 12 wherein the milk thistle extract is present in the solid composition.

22. The skin care formulation according to claim 21 wherein the milk thistle extract is present in an amount of from about 30 to about 90 wt % of the solid composition.

23. The skin care formulation according to claim 12 wherein the vitamin E or derivative thereof is present in the solid composition.

24. The skin care formulation according to claim 23 wherein the vitamin E or derivative thereof is present in an amount of from about 5 to about 50 wt % of the solid composition.

25. The skin care formulation according claim 1 further comprising a liquid vehicle.

26. The skin care formulation according to claim 25 wherein the liquid vehicle is water.

27. The skin care formulation according to claim 25 wherein the liquid vehicle is at least about 80 wt % of the skin care formulation.

28. The skin care formulation according to claim 25 wherein the formulation is a beverage for oral administration.

29. The skin care formulation according claim 12 further comprising a capsule.

30. The skin care formulation according to claim 29 wherein the capsule comprises dandelion root extract, horse chestnut extract, L-cysteine or a derivative thereof, copper or a salt thereof, red clover extract, ascophyllum kelp, or combinations thereof.

31. A method of treating cellulite comprising administering an effective amount of the skin care formulation according claim 1 to a subject in need thereof.

32. The method of claim 31 wherein the formulation is a beverage according to claim 28 and where the effective amount of the beverage is from about 50 to about 300 mL/day.

33. The method of claim 31 wherein the skin care formulation is administered daily for at least about 30 days.
